(19) United States
(12) Patent Application Publication
(10) Pub. No.: US 2019/0290880 A1
(43) Pub. Date: Sep. 26, 2019

(54) POSITIVE AIRWAY PRESSURE OIL DELIVERY SYSTEM

(71) Applicant: Ideya Labs, LLC, Orem, UT (US)

(72) Inventors: Thomas D. Dickson, JR., Orem, UT (US); Steven K. Atkinson, Lehi, UT (US); Gregory D. Jackson, Bountiful, UT (US); Kathryn Dickson, Orem, UT (US)

(73) Assignee: Ideya Labs, LLC, Orem, UT (US)

(21) Appl. No.: 16/359,043

(22) Filed: Mar. 20, 2019

Related U.S. Application Data
(60) Provisional application No. 62/645,757, filed on Mar. 20, 2018.

(51) Int. Cl. A61M 16/14; A61M 16/06; A61M 16/00; A61M 16/08; A61M 21/00

(52) U.S. Cl. CPC A61M 16/142; A61M 16/06; A61M 16/0057; A61M 2205/123; A61M 21/00; A61M 2021/0016; A61M 2202/0468; A61M 16/0875

(57) ABSTRACT

This disclosure describes a passive inline oil delivery system for delivering a controlled and variable amount of an oil, such as an aromatic or essential oil, to a user via a positive airway pressure system. A positive airway pressure system may include a tube that delivers pressurized air to a user during, for example, sleep. Examples of such positive airway pressure systems include continuous positive airway pressure (CPAP) systems, automatic positive airway pressure (APAP) systems, and bilevel positive airway pressure (BiPAP) systems. The inline passive oil delivery system described herein delivers a controlled and/or adjustable amount of oil into the air delivered by the positive airway pressure system to the user.

### RELATED APPLICATION

This claims the benefit of U.S. Provisional App. No. 62/645,757 filed on 20 Mar. 2018, now pending, the disclosure of which is incorporated, in its entirety, by this reference.

### TECHNICAL FIELD

The present disclosure generally relates to oil delivery systems for positive airway pressure systems. Specifically, this disclosure describes a passive inline oil delivery system for delivering a controlled and variable amount of a chemical compound, such as an aromatic or essential oil, via a positive airway pressure system.

### BRIEF DESCRIPTION OF THE DRAWINGS

The written disclosure herein describes illustrative embodiments that are non-limiting and non-exhaustive. Reference is made to certain of such illustrative embodiments that are depicted in the figures described below.

FIG. 1 illustrates a positive airway pressure system providing pressurized airflow via a tube to a facemask worn by a user.

FIG. 2 illustrates three example locations for inserting a passive, inline oil delivery system, according to various embodiments.

FIG. 3 illustrates a passive, oil delivery system connected inline proximate the facemask of the positive airway pressure system.

FIG. 4A illustrates a tube-side view of a first portion of an example passive oil delivery system, according to one embodiment.

FIG. 4B illustrates a connector-side view of the first portion of the example passive oil delivery system, according to one embodiment.

FIG. 5A illustrates a tube-side view of a second portion of the example passive oil delivery system, according to one embodiment.

FIG. 5B illustrates a connector-side view of the second portion of the example passive oil delivery system, according to one embodiment.

FIG. 6 illustrates the example passive oil delivery system in a disassembled state, including an example diffusion pad along with the connecter-side views of the first and second portions, according to one embodiment.

FIG. 7 illustrates the first and second portions of the passive oil delivery system joined together in a “fully open” setting, according to one embodiment.

FIG. 8 illustrates a cut-away view of the passive oil delivery system showing the partially exposed adjustable pad housing, according to one embodiment.

FIG. 9 illustrates two additional adjustment settings for the passive oil delivery system in dashed lines, according to one embodiment.

FIG. 10 illustrates an alternative passive oil delivery system that is circular and allows for rotational adjustment of the first and second portions to adjust the amount of internal pad exposure, according to one embodiment.

FIGS. 11A-11B illustrate example embodiments of passive oil delivery systems that are preconfigured with fixed delivery rates, according to various embodiments.

### SUMMARY

According to one aspect of the present disclosure, an oil diffusing apparatus to expose oil contained in a holder to an airflow of a positive airway pressure system is provided. The oil diffusing apparatus may comprise a receptacle configured to secure a holder containing the oil, and a first housing portion and a second housing portion, the first and second portions coupled to each other to form a housing through which an airflow may pass. An aperture may be formed in the receptacle to expose the holder containing the oil to the airflow, the aperture being variable in size to change the amount of oil carried by the airflow.

The first portion and the second portion may be capable of entirely enclosing the holder in response to being joined, such that no part of the holder is exposed to the airflow. The oil diffusing apparatus may be configured to be inserted between a tube and a facemask or between a tube and a pump of the positive airway pressure system.

In some embodiments, a user may select a degree of exposure of the holder by adjusting the coupling of the first portion with the second portion. The holder may comprise an absorbent material, atomizing material, nebulizing material, felt pad, plant-based fiber pad, animal-based fiber pad, fabric, gel, porous substance, plastic, or sponge. An exterior of the second portion may comprise one or more detents that are configured to contact one or more protrusions located on an interior of the first portion. The second portion may comprise measurement indicators that correspond to an amount of exposure of the holder. The first portion and the second portion may be coupled via a threaded engagement.

In another aspect of the present disclosure, an oil diffusing apparatus to expose oil contained in a holder into an airflow of a positive airway pressure system is provided. The apparatus may comprise a receptacle configured to receive the holder containing the oil, and a cover coupled to the receptacle to form a housing for the holder, the cover being movable relative to the receptacle to provide an aperture to vary exposure of the holder to the airflow.

The size of the aperture may correspond to the exposure of the holder to the airflow. A user may select a degree of exposure of the holder by adjusting the size of the aperture. The receptacle may move relative to the cover to entirely close the aperture such that no part of the holder is exposed to the airflow.

The apparatus may be configured to be inserted between a tube and a facemask or between a tube and a pump of the positive airway pressure system. The holder may comprise an absorbent material, atomizing material, nebulizing material, felt pad, plant-based fiber pad, animal-based fiber pad, fabric, gel, porous substance, plastic, or sponge. The apparatus may comprise measurement indicators that correspond to an amount of exposure of the holder.

In another aspect of the present disclosure, a method for diffusing an adjustable amount of oil into a positive airway pressure system is set forth. The method may comprise inserting a passive oil delivery apparatus in-line with an airflow of the positive airway pressure system, the passive oil delivery apparatus comprising an oil-soaked material disposed in a receptacle and adjustably covered with a cover, and adjusting the cover such that a portion of the oil-soaked material is exposed to the airflow.

The above summary of the present invention is not intended to describe each embodiment or every implementation of the present invention. The Figures and the detailed description that follow more particularly exemplify a preferred embodiment.

### DETAILED DESCRIPTION

Many people suffer from sleep apnea and other sleeping disorders and can benefit from the use of positive airway pressure systems. Positive airway pressure systems, such as continuous positive airway pressure (CPAP) systems, automatic positive airway pressure (APAP) systems, and bilevel positive airway pressure (BiPAP) systems deliver pressurized air to a user via a tube and a face mask. The face mask may deliver air to the nose and/or mouth of a user during, for example, sleep. The presently described systems and methods may be adapted for use with a wide variety of alternative types of air or other gas delivery systems.

Aromatic essential oils are used for aromatherapy, massage therapy, nutritional supplements, personal care, medicinal use, and the like. Some systems and devices have been devised to work with CPAP machines to deliver essential oils to users. For example, U.S. Patent Publication No. 2018/0008796 describes an inline scent delivery adaptor for CPAP that passively diffuses oil from a pad into air delivered to the CPAP user. However, the scent delivery adaptor of the above-referenced patent application does not offer a controlled delivery of the oil into the airflow nor does it allow for any adjustability. Instead, the existing scent delivery adaptor diffuses an uncontrollable, large amount of oil into the airflow until the oil reserves are exhausted.

Similarly, an apparatus for diffusing aromatic substances in ventilatory equipment is described in U.S. Patent Publication No. 2010/0022819. This system likewise lacks any ability to control or adjust the amount of oil diffused into the airflow. The presently described systems and apparatuses allow for controlled and/or adjustable diffusion (or infusion) of oils, such as essential or aromatic oils, into the pressurized delivery tube(s) of positive airway pressure systems. The presently-described system and apparatuses are passively connected inline to the tube(s) of the positive airway pressure system. It will be understood that the presently described system can operate with not only essential or aromatic oils, but with any chemical compounds that can evaporate into the air. Oil, as used herein, is intended to cover a broad spectrum of chemical compounds.

In some embodiments, an inline passive oil delivery system provides a controlled, time-release delivery of an oil into the airflow of a positive airway pressure system. For example, a saturated or partially saturated pad may store a reserve of oil. The inline passive oil delivery system may expose a portion of the pad to the airflow within the tube. The exposed portion of the pad may diffuse, or possibly infuse (e.g., via capillary action), essential oils into the airflow at a controlled rate. The pad type and the area of the pad exposed to the airflow may be selected to provide a target diffusion (or infusion) rate of an essential oil.

In one specific example, a pad is selected to hold approximately five hundred microliters (500 μl) of an essential oil corresponding to approximately 10 “drops” of an essential oil. The pad is contained within an apparatus connected inline with the tubes of a positive airway pressure system. A portion of the pad may be exposed to the airflow to deliver, for example, 5 μl of oil into the airflow per hour (approximately 0.1 “drops” per hour).

In another specific example, a larger portion of the pad may be exposed to the airflow and/or a pad having a higher release rate may be utilized. In such an example, the pad may deliver 10 μl of oil into the airflow per hour (approximately 0.2 “drops” per hour). The area of the pad exposed to the airflow and the type of pad utilized may be selected to attain a target delivery rate. Example target delivery rates may range from 1-100 μl per hour. Higher and lower rates are functionally possible by exposing more or less of the pad and/or by selecting different pad materials or pad saturation.

A pad (or other material) may be configured to store and provide a controlled release, such as a constant release over time (e.g., a zero-order release) of any of a wide variety of oils, including aromatic oils, scents, essential oils, and the like. Extracts and absolutes may also be used instead of true “essential oils” that are manufactured via steam distillation, expression, or cold-distillation. Such oils may provide pleasuring or calming scents, may open airways to improve breathing quality, and/or may provide other therapeutic or non-medical device medicinal benefits to a user. In some embodiments, a cannabis oil may be added to the pad for controlled and/or adjustable diffusion/infusion of cannabis oil into a breathing tube of a user.

In some embodiments, the pad may be replaced by any of a wide variety of materials and substances capable of storing a reserve of oil and delivering the oil into an airflow. Some materials may diffuse oil into the airflow via, for example, evaporation or atomization, and other materials may infuse oil into the airflow via, for example, capillary actions. Examples of possible materials include, but are not limited to, atomizing materials, nebulizing materials, felt pads, plant-based fiber pads, animal-based fiber pads, fabrics, gels, porous substances, plastics, sponges, and the like. In some embodiments, a container may be used that comprises a material that is impermeable to liquid while still allowing gas to pass through. In one specific embodiment, the pad may comprise compressed felt that is between approximately 0.0625 and 0.5 inches thick (1.5 mm to 13 mm), between approximately 0.2 and 0.75 inches wide (5 mm to 20 mm), and between approximately 0.2 and 1.125 inches long (5 mm to 30 mm).

In some embodiments, the inline apparatus that contains the pad is mechanically adjustable. Mechanical adjustment of the apparatus can adjust the total surface area of the pad (or other material) that is exposed to the airflow. In some embodiments, the inline apparatus may be adjusted between an “off” setting and a “fully open” setting. The inline apparatus may be infinitely adjustable between the “off” and “fully open” settings. Alternatively, the inline apparatus may have a preset number of stepped adjustment settings between the “off” and “fully open” settings.

In the “off” setting, no portion of the pad may be exposed to the airflow and the rate of release of the oil into the airflow may be approximately zero (0 μl). The “fully open” setting may correspond to a maximum number of drops or microliters per hour. For example, the “fully open” setting may correspond to 1, 2, or 3 “drops” per hour. Thus, in an example embodiment in which there are 4 discrete stepped settings between “off” and a “fully open” setting of 1 “drop” per hour, the discrete steps may correspond to release rates of 0.2, 0.4, 0.6, and 0.8 “drops” per hour.

Thus, in some embodiments, an inline oil delivery system may be configured to deliver a preset number of “drops” per hour of an oil into an airflow of a positive airway pressure system. In other embodiments, the inline oil delivery system may be mechanically adjustable to control the number of “drops” per hour of the oil into the airflow of the positive airway pressure system.

In various embodiments, the pad (or other oil storage and infuser/diffuser material) may be washable and/or replaceable. In some embodiments, the pads or other materials may come preloaded with specific oils. In other embodiments, the user may add oil to the bare pad prior to use. In some embodiments, the pad may be configured to change color when oil is added (e.g., via a chemical reaction). In some embodiments, the color of a pad may fade or change color over time to indicate that the pad should be replaced.

The material from which the pad or other release mechanism is selected may be configured to provide a zero-order release. That is, the amount of oil released per hour may be substantially the same from initial release until the stored oil supply within the pad is exhausted. In some embodiments, the pad may release a slightly greater amount of oil early on (to satisfy the expectations of the user early on) and then taper off slightly to a zero-order or near zero-order delivery rate for the remaining delivery time.

The embodiments of the disclosure can be further understood by reference to the drawings of some specific example embodiments, wherein like parts are designated by like numerals throughout. The components of the disclosed embodiments, as generally described and illustrated in the figures herein, could be arranged and designed in a wide variety of different configurations. Thus, the following description of the embodiments of the systems and methods of the disclosure is not intended to limit the scope of the disclosure, as claimed, but is merely representative of possible embodiments.

It is particularly appreciated that many of the components could be resized, reshaped, lengthened, shortened, etc. It is also appreciated that a wide variety of connections, couplings, and fasteners could be utilized in addition to, or as alternatives to, those shown in the figures. In fact, many possible options and variations are intentionally not illustrated to avoid obscuring other aspects of the illustrated embodiments.

The various components described herein may be manufactured using a wide variety of metals, plastics, glasses, woods, and other materials known to be useful in manufacturing. In some cases, well-known structures, materials, or operations may not be shown or described in detail in order to avoid obscuring aspects of the disclosure. Furthermore, the described features, structures, or characteristics may be combined in any suitable manner in one or more alternative embodiments.

It is appreciated that various mechanical interfaces may be substituted with alternative mechanical components that provide a similar function. For example, press-fit interference fittings may be replaced with screw fittings, glued fittings, clamps, and the like. Detents, protrusions, depressions, snap fittings, press-fittings, rubber seals, screw fittings, interference fits, and the like may be used interchangeably and/or in combination to accomplish the described functions.

The phrases “connected to,” “coupled to,” “engage with,” and “in communication with” refer to any form of interaction between two or more components, including mechanical, electrical, magnetic, and electromagnetic interaction, depending on the context. Two components may be connected to each other, even though they are not in direct contact with each other, and even though there may be intermediary devices between the two components.

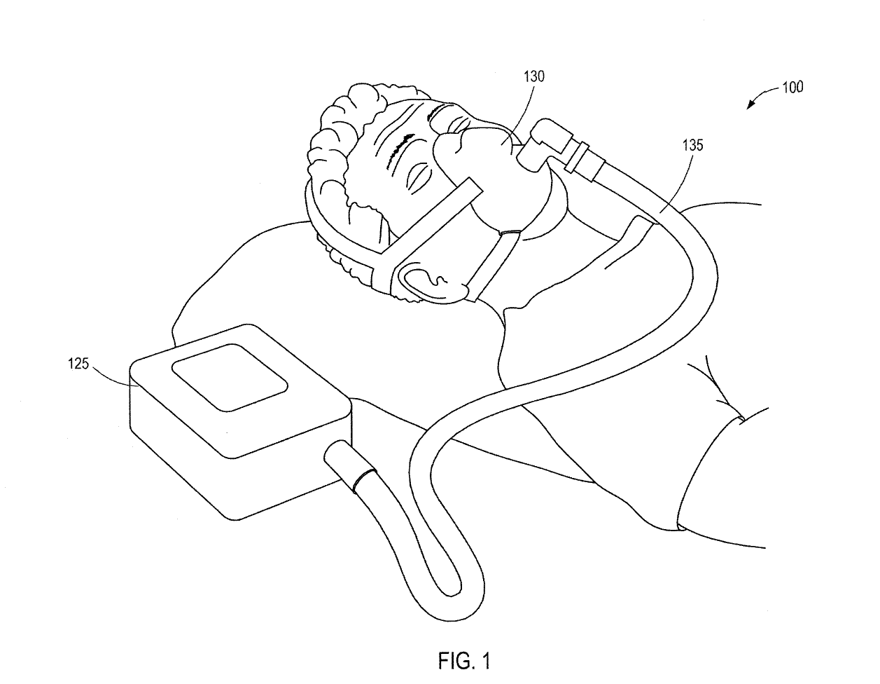

FIG. 1 illustrates a positive airway pressure system 125 providing pressurized airflow via a tube 135 to a facemask 130 worn by a user 100. The positive airway pressure system 125 may, for example, be a continuous positive airway pressure (CPAP) system, automatic positive airway pressure (APAP) system, or a bilevel positive airway pressure (BiPAP) system. The tube 135 may be connected to the facemask 130 via a mechanical coupler. Similarly, the tube 135 may be connected to the positive airway pressure system 125 via a mechanical coupler. In some embodiments, the tube 135 may be a single piece. In other embodiments, the tube 135 may comprise multiple pieces mechanically coupled together.

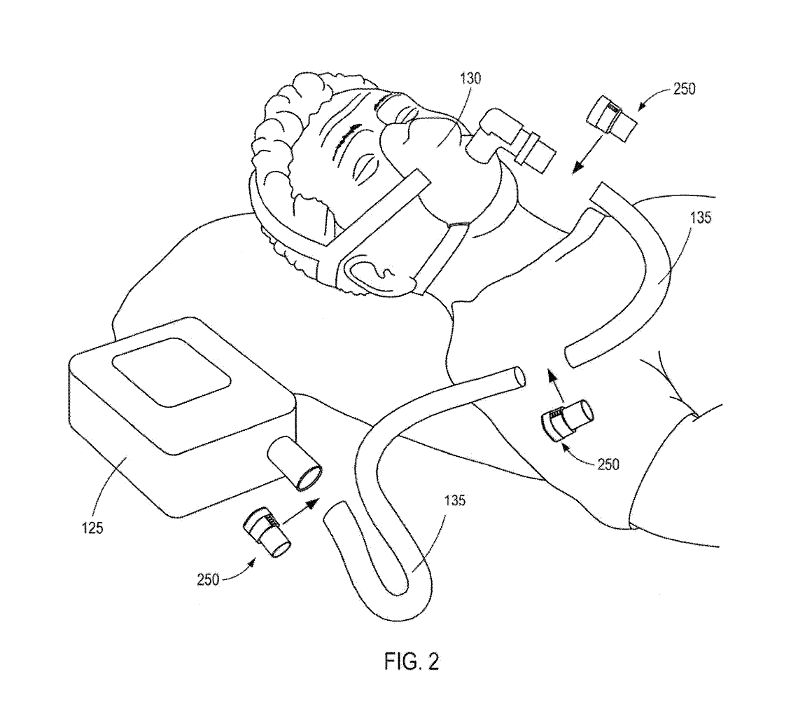

FIG. 2 illustrates three example locations for inserting a passive, inline oil delivery system 250, according to various embodiments. More than one inline oil delivery system 250 may be inserted. In one embodiment, the inline oil delivery system 250 may be inserted between a mechanical coupler of the positive airway pressure system 125 and the tube 135. In another embodiment, the inline oil delivery system 250 may be inserted between two sections of the tube 135. In yet another embodiment, the inline oil delivery system 250 may be inserted between the tube 135 and the facemask 130.

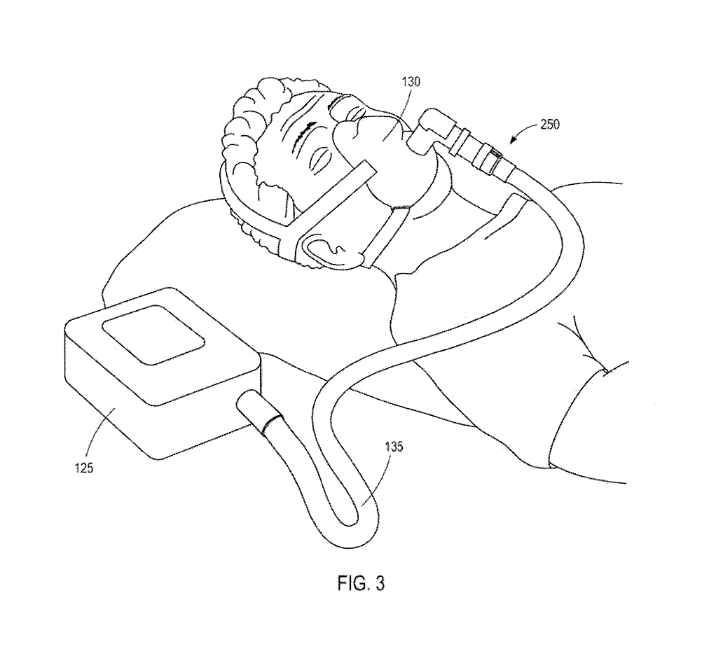

FIG. 3 illustrates a passive oil delivery system 350 connected inline with the tube 135 proximate the facemask 130 of the positive airway pressure system 125.

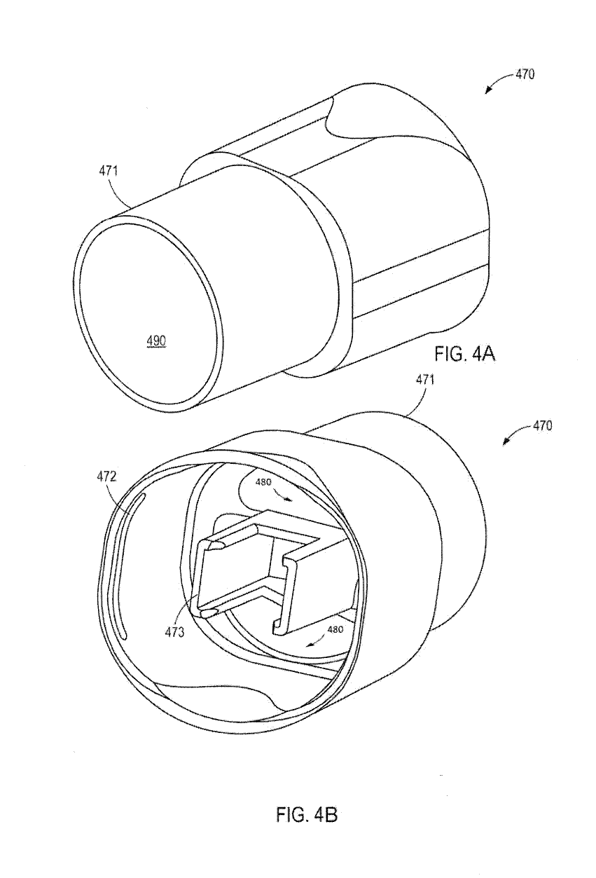

FIG. 4A illustrates a tube-side view of a first portion 470 and FIG. 4B illustrates a connecter-side view of the first portion 470 of an example passive oil delivery system 450, according to one embodiment. As illustrated, airflow may pass through a tube connector 490 into the first portion 470. Apertures 480 may facilitate passage of the airflow around a pad holder 473. A tube fitting 471 may provide a friction fit within or around the tube of a positive airway pressure system. The pad holder 473 may be configured to hold a felt pad that stores reserves of oil.

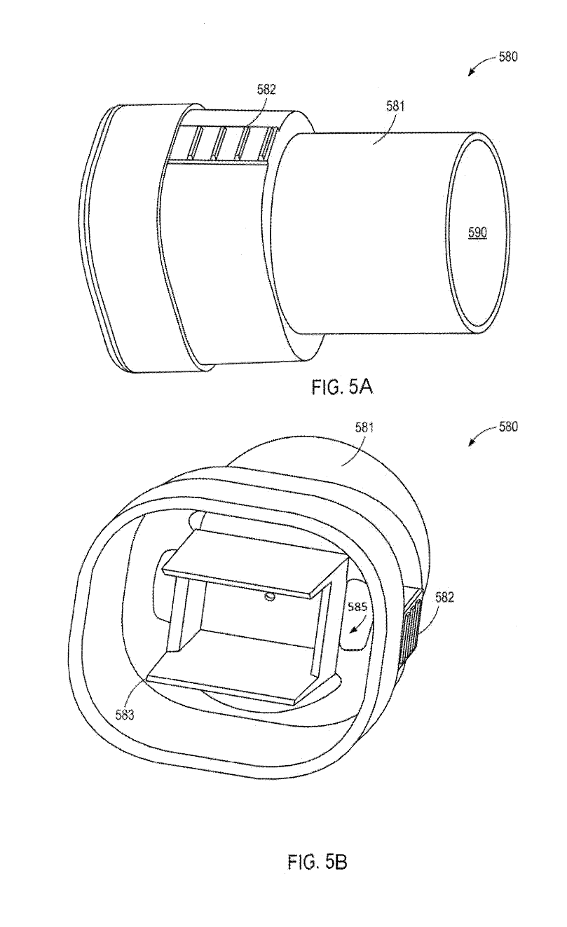

In various embodiments, the pad holder 473 may be replaced with various other components to hold atomizing materials, nebulizing materials, felt pads, plant-based fiber pads, animal-based fiber pads, fabrics, gels, porous substances, plastics, sponges, and the like. The first portion 470 of the passive oil delivery system may include one or more protrusions 472 for interacting with detents on a second portion of the passive oil delivery system shown in FIGS. 5A and 5B.

In some embodiments, the length of the pad selected to extend from the pad holder 473 dictates or at least partially dictates the delivery rate of the oil. Similarly, the shape of the pad holder 473 or the amount of surface area of the pad holder 473 may impact the delivery rate (e.g., holes in the walls of the pad holder 473 may increase the delivery rate).

FIG. 5A illustrates tube-side view and FIG. 5B illustrates a connecter-side view of the second portion 580 of the example passive oil delivery system, according to one embodiment. A tube fitting 581 may provide a friction fit within or around the tube or facemask of the positive airway pressure system. Detents 582 can be configured to interact with the protrusion(s) 472 on the first portion 470 of the passive oil delivery system as the second portion 580 is inserted within the first portion. Detents 582 may be present on one or more sides of the second portion 580 of the passive oil delivery system to interact with protrusions 472 on opposing inner sides of the first portion 470 of the passive oil delivery system.

In some embodiments, the second portion 580 of the passive inline oil delivery system may include a pad cover 583. In other embodiments, the pad cover 583 may not be present such that the configuration of the pad holder 473, the dimensions of the pad, and the pad material utilized may dictate the delivery rate of the oil. The pad cover 583 may selectively interact with the pad holder 473 of the first portion 470 to expose varying amounts of the secured pad to the airflow through the tube connector 590. The engagement between the pad holder 473 and the pad cover 583 may be capable of forming an airtight volume in which the pad is contained. In some embodiments, the pad holder 473 and the pad cover 583 may form a space that is smaller than the pad, such that the pad can be compressed as the first portion 470 and second portion 580 are brought together.

Apertures 585 may facilitate the passage of airflow through the second portion 580. The apertures 585 may be shaped and positioned to align with corresponding apertures 480 in the first portion 470. The joined first portion 470 and second portion 580 can form a conduit through which the airflow passes (e.g., through apertures 480, 585.

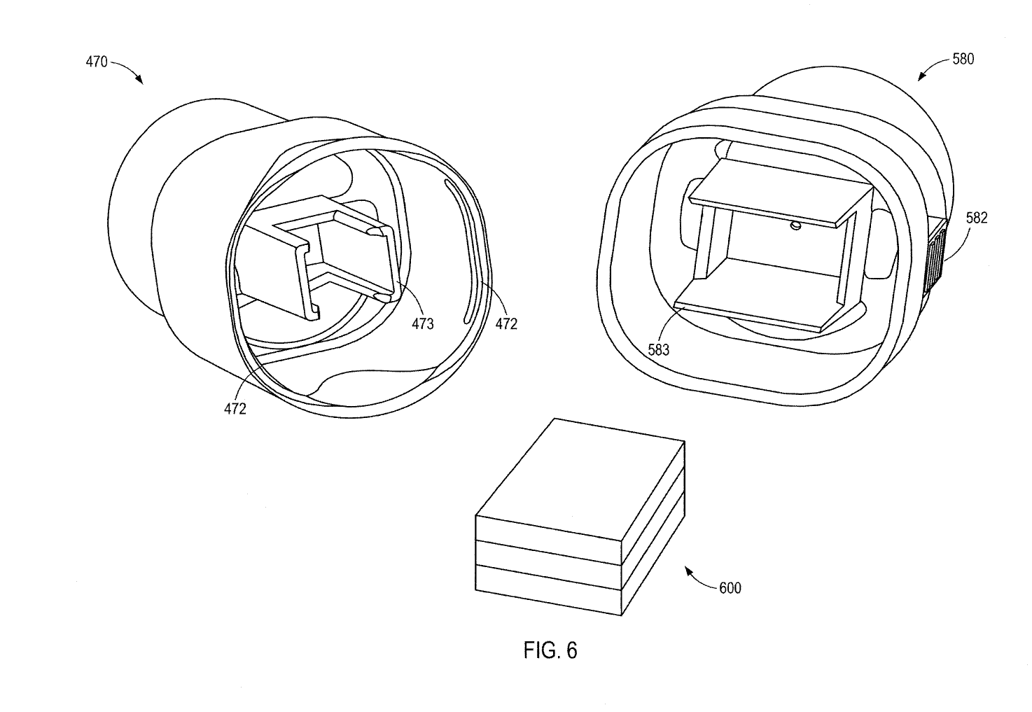

FIG. 6 illustrates an example diffusion pad 600 along with the connecter-side views of the first portion 470 and the second portion 580 of the example passive oil delivery system, according to one embodiment. The essential oils, aromatic oils, or the like may be added to the diffusion pad 600. The diffusion pad 600 may comprise a single pad or multiple layers of one or more material types. As previously noted, the pad 600 may be replaced by various alternative materials for retaining and time-releasing an oil, including various nebulizing materials, felt pads, plant-based fiber pads, animal-based fiber pads, fabrics, gels, porous substances, plastics, sponges, and the like.

In the illustrated embodiment, the pad 600 is ready to be inserted within the pad holder 473 of the first portion 470 of the passive oil delivery system. As the second portion 580 of the passive oil delivery system is inserted within the first portion 470, the protrusions 472 may be fitted between one of a plurality of detents 582. Depending on how far the second portion 580 is inserted within the first portion 470, the pad 600 may be exposed to the airflow by varying degrees.

For example, in a fully closed or “off” position, the second portion 580 may be inserted within the first portion 470 until the pad cover 583 covers all of the portions of the pad 600 that would otherwise be exposed to the airflow by the pad holder 473. In a fully open position, the second portion 580 may only be inserted partway (e.g., until the protrusions 472 are between the first two detents 582 of the second portion 580) such that the pad holder 473 and pad cover 583 act to expose a portion of the pad 600 to the airflow through the passive oil delivery system. In the “fully open” configuration, the pad 600 may be entirely exposed to the airflow, with the exception of those portions of the pad 600 that are covered by the pad holder 473.

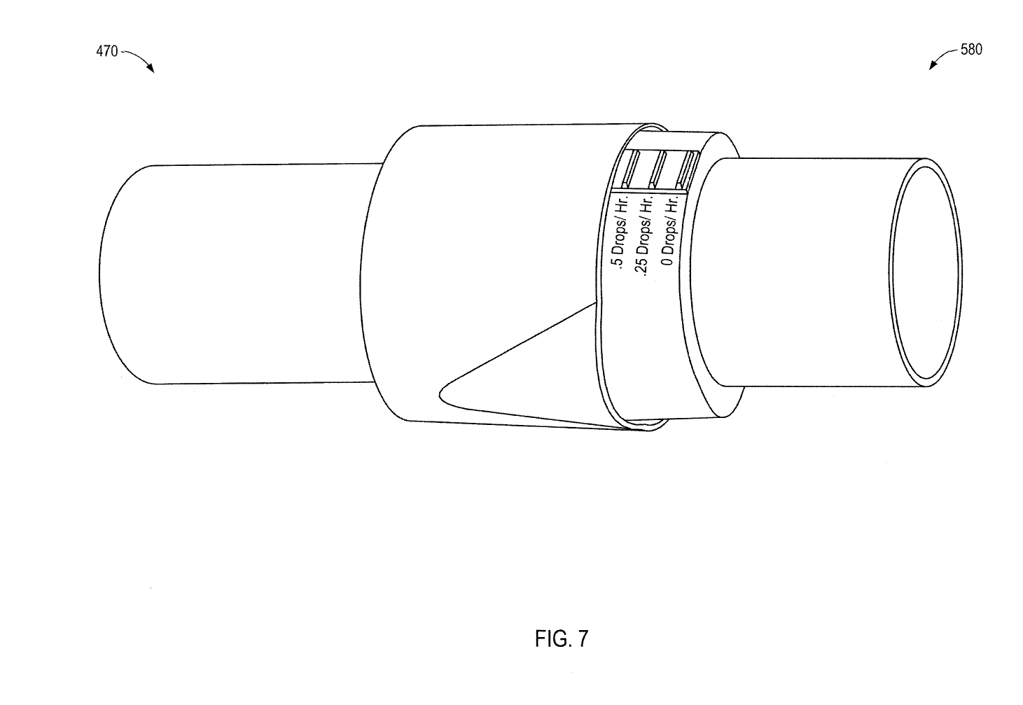

FIG. 7 illustrates the first portion 470 and second portion 580 of the passive oil delivery system joined together in a “fully open” setting, according to one embodiment. In the illustrated embodiment, a portion of the pad may be exposed to the airflow within the passive oil delivery system to allow for approximately 0.5 “drops” (approximately 25 μl) to be diffused or infused into the airflow per hour. As illustrated, by pushing the second portion 580 further into the first portion 470, the diffusion rate may be decreased to 0.25 “drops” per hour or turned off completely.

In an embodiment in which the pad can store 15 “drops” of, for example, a rosemary essential oil, the user may set the passive oil delivery system to deliver 0.25 “drops” of oil per hour while he or she sleeps for eight hours. Approximately 2 “drops” of oil may be infused (or diffused) into the airflow during the eight-hour sleep session. The remaining 13 “drops” may be conserved during the day by pushing the second portion 580 further into the first portion 470 to the “0 drops/hour” mark to prevent diffusion of any of the stored rosemary essential oil into the air in the tube of the positive airflow pressure system. When the user retires the following evening, he or she may separate the first 470 and second 580 portions to the desired delivery rate (infusion or diffusion rate). As depicted, the protrusions and detents may correspond to a given delivery rate.

In some embodiments, the system can include an automated delivery system. For instance, an electric motor can control the relative motion of the first portion 470 and the second portion 580 to vary the degree of exposure of the pad. A user may operate the electric motor via a remote or controls located on the system. In some embodiments, the automated system can implement a timer or schedule for automatically adjusting the exposure of the pad. In some embodiments, the system may be capable of detecting when a user falls asleep and in response may be programmed to close off the pad.

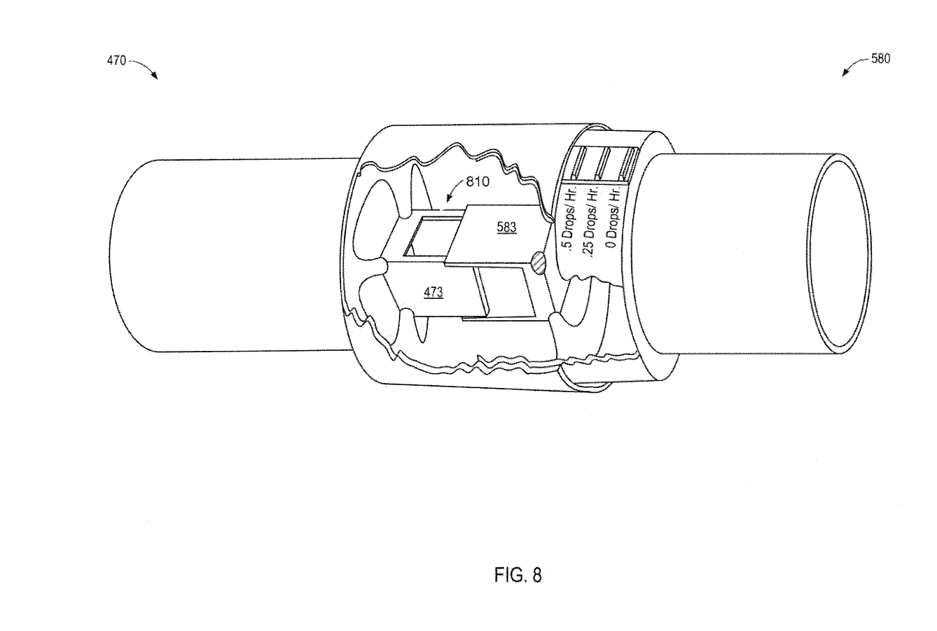

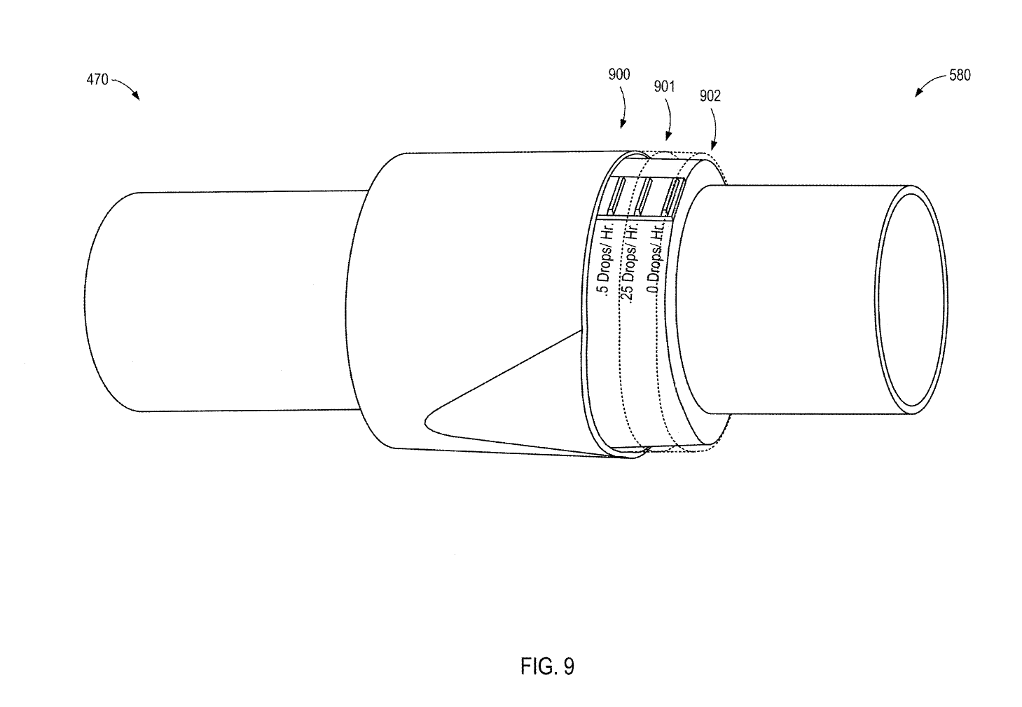

FIG. 8 illustrates a cut-away view of the passive oil delivery system showing the location of where a partially exposed pad (removed from the figure for clarity) would be positioned within the pad holder 473 and the pad cover 583. As can easily be visualized in the context of FIG. 8, pushing the second portion 580 further into the first portion 470 will cause the pad cover 583 to further cover a pad secured within the pad holder 473. Likewise, as the first portion 470 and the second portion 580 are separated, an aperture 810 is created between the pad holder 473 and the pad cover 583. A variety of aperture shapes and sized are possible depending on the configuration of the pad holder 473 and pad cover 583 and also depending on the mechanism/motion by which the first portion 470 and the second portion 580 couple. For instance, in the illustrated embodiment, the aperture 810 allows for a multi-sided exposure of the pad. Such a configuration may be desirable to better diffuse the oil into the airflow. FIG. 9 illustrates three example adjustment settings for the passive oil delivery system, according to one embodiment. A first position 900 corresponds to 0.5 “drops” per hour, a second position 901 corresponds to 0.25 “drops” per hour, and 0 “drops” per hour in a third position 902. In some embodiments, additional detents may be present corresponding to additional delivery rates. For example, eleven settings may allow for delivery rates between 0 and 2 “drops” per hour in 0.2 “drops” per hour increments.

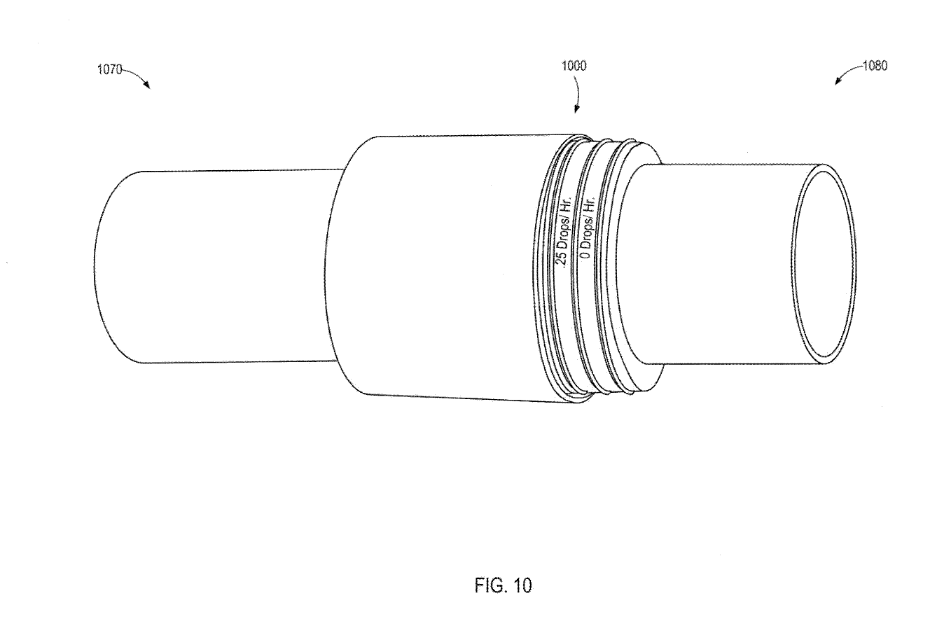

FIG. 10 illustrates an alternative passive oil delivery system that is circular (instead off the substantially oval or rounded-rectangular embodiments previously illustrated). The illustrated circular passive oil delivery system comprises a first portion 1070 and a second portion 1080 that are rotationally threaded together to select a target delivery rate, at 1000. In other embodiments, the first and second portions may be press fit, include rubber (or similar) seals, or utilize a wide variety of alternative mechanical couplings known in the art. It will be understood that for each different mechanism for coupling the first portion and the second portion, it may be required to change the shapes of the pad holder and pad cover and also the way in which the pad holder and the pad cover engage. For instance, in the embodiment illustrated in FIG. 10, in which the first portion 1070 and the second portion 1080 are rotationally threaded together, the pad holder and pad cover may have cylindrical exteriors configured to fit together to form one or more exposure areas as the first portion 1070 and the second portion 1080 are rotated apart.

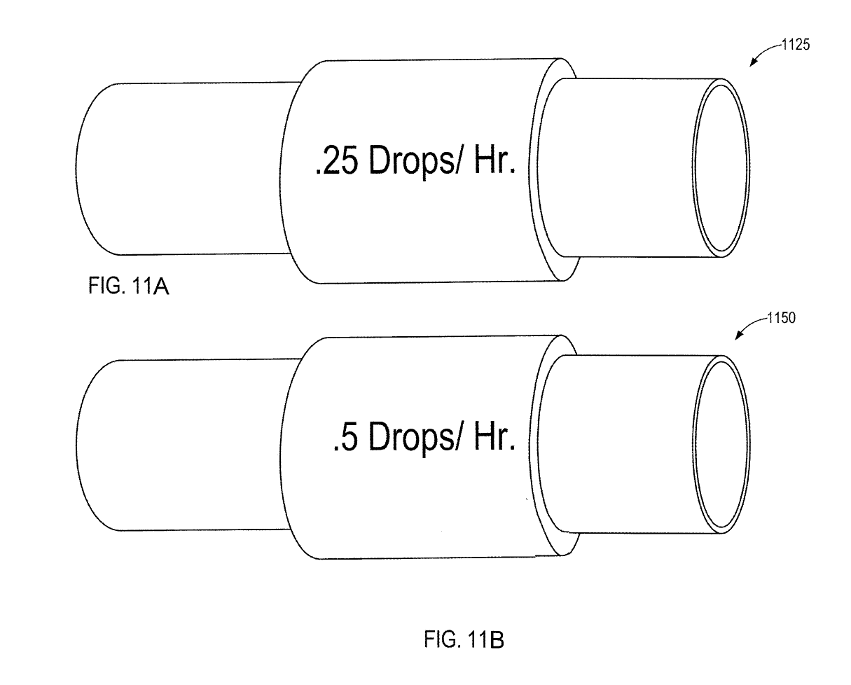

FIGS. 11A and 11B illustrate example embodiments of passive oil delivery systems that are preconfigured with fixed delivery rates, according to various embodiments. FIG. 11A illustrates an inline passive oil delivery system 1125 that is preconfigured with a pad type and exposure to the airflow to allow for a delivery rate of 0.25 “drops” per hour. FIG. 11B illustrates an inline passive oil delivery system 1150 that is preconfigured with a different pad type and/or a different (i.e., greater) exposure area to the airflow to allow for a delivery rate of 0.5 “drops” per hour.

Specific embodiments and applications of the disclosure are described above and illustrated in the figures. It is, however, understood that many adaptations and modifications can be made to the precise configurations and components detailed above. Again, in some cases, well-known features, structures, or operations are not shown or described in detail. Furthermore, the described features, structures, or operations may be combined in any suitable manner in one or more embodiments. It is also appreciated that the components of the embodiments as generally described and illustrated in the figures herein could be arranged and designed in a wide variety of different configurations. That is, all feasible permutations and combinations of embodiments are contemplated.

In the description above, various features are sometimes grouped together in a single embodiment, figure, or description thereof for the purpose of streamlining the disclosure.

It will be apparent to those having skill in the art that changes may be made to the details of the above-described embodiments without departing from the underlying principles of the invention.

What is claimed is:

1. An oil diffusing apparatus to expose oil contained in a holder to an airflow of a positive airway pressure system, the oil diffusing apparatus comprising:
a receptacle configured to secure a holder containing oil;
a first housing portion and a second housing portion, the first and second portions coupled to each other to form a housing;
an airflow passing through the housing;
an aperture formed in the receptacle to expose the holder containing the oil to the airflow, the aperture being variable in size to change the amount of oil carried by the airflow.

2. The apparatus of claim 1, wherein the first portion and the second portion are capable of entirely enclosing the holder in response to being joined, such that no part of the holder is exposed to the airflow.

3. The apparatus of claim 1, wherein the oil diffusing apparatus is configured to be inserted between a tube and a facemask of the positive airway pressure system.

4. The apparatus of claim 1, wherein the oil diffusing apparatus is configured to be inserted between a tube and a pump of the positive airway pressure system.

5. The apparatus of claim 1, wherein a user can select a degree of exposure of the holder by adjusting the coupling of the first portion with the second portion.

6. The apparatus of claim 1, wherein the holder comprises an absorbent material, atomizing material, nebulizing material, felt pad, plant-based fiber pad, animal-based fiber pad, fabric, gel, porous substance, plastic, or sponge.

7. The apparatus of claim 1, wherein an exterior of the second portion comprises one or more detents that are configured to contact one or more protrusions located on an interior of the first portion.

8. The apparatus of claim 1, wherein the second portion comprises measurement indicators that correspond to an amount of exposure of the holder.

9. The apparatus of claim 1, wherein the first portion and the second portion are coupled via a threaded engagement.

10. An oil diffusing apparatus to expose oil contained in a holder into an airflow of a positive airway pressure system, the apparatus comprising:
a receptacle configured to receive the holder containing oil; and
a cover coupled to the receptacle to form a housing for the holder, the cover being movable relative to the receptacle to provide an aperture to vary exposure of the holder to the airflow.

11. The apparatus of claim 10, wherein the size of the aperture corresponds to the exposure of the holder to the airflow.

12. The apparatus of claim 10, wherein a user can select a degree of exposure of the holder by adjusting the size of the aperture.

13. The apparatus of claim 10, wherein the receptacle can move relative to the cover to entirely close the aperture such that no part of the holder is exposed to the airflow.

14. The apparatus of claim 10 configured to be inserted between a tube and a facemask of the positive airway pressure system.

15. The apparatus of claim 10 configured to be inserted between a tube and a pump of the positive airway pressure system.

16. The apparatus of claim 10, wherein the holder comprises an absorbent material, atomizing material, nebulizing material, felt pad, plant-based fiber pad, animal-based fiber pad, fabric, gel, porous substance, plastic, or sponge.

17. The apparatus of claim 10, wherein the apparatus comprises measurement indicators that correspond to an amount of exposure of the holder.

18. A method for diffusing an adjustable amount of oil into a positive airway pressure system, the method comprising:
inserting a passive oil delivery apparatus in-line with an airflow of the positive airway pressure system, the passive oil delivery apparatus comprising an oil-soaked material disposed in a receptacle and adjustably covered with a cover; and
adjusting the cover such that a portion of the oil-soaked material is exposed to the airflow.
